(19) United States
(12) Patent Application Publication
(10) Pub. No.: US 2016/0253687 A1
(43) Pub. Date: Sep. 1, 2016

(54) SYSTEM AND METHOD FOR PREDICTING HEALTHCARE COSTS

(71) Applicant: Aetna Inc., Hartford, CT (US)

(72) Inventors: Henry G. WEI, Larchmont, NY (US); Gregory B. STEINBERG, Dingmans Ferry, PA (US); Lonny Reisman, Glen Head, NY (US)

(21) Appl. No.: 14/632,886

(22) Filed: Feb. 26, 2015

(51) Int. Cl. G06Q 30/02; G06F 17/30; G06Q 50/22; G06F 19/00

(52) U.S. Cl. CPC G06Q 30/0202; G06F 19/322; G06F 17/30917; G06F 17/30867; G06F 19/327; G06F 19/3431; G06Q 50/22

(57) ABSTRACT

Predicting future healthcare costs is disclosed. A method includes: receiving medical care information (e.g., claims data) relating to patients; identifying, by a cost prediction module, a presence of one or more monitored events representing modifiable health risks when the claims data indicates that the patient is receiving medical care that is not in compliance with evidence-based medical standards; mapping, by the cost prediction module, each of the one or more monitored events to utilization and/or cost prediction values based on a status of each of the one or more monitored events; assigning, by the cost prediction module, for each of the one or more monitored events, a probability score for utilization of medical services based on the status of the monitored event; and calculating a prediction of future healthcare utilization and/or cost based on the probability score and status for each of the one or more monitored events.

### TECHNICAL FIELD

This disclosure relates generally to the field of health care management and more specifically to the area of predicting future healthcare costs.

### BACKGROUND

In conventional approaches, predictive models have been used to predict healthcare utilization and costs based on static factors—e.g., age, gender, diagnoses, and medications of individuals in the populations used to develop the models. Such models have achieved modest accuracy. However, these conventional approaches do not always generate accurate predictions, in part based on the coarseness of the predictive variables models used, which may not account for the quality of care of a medical condition, as opposed to the severity or presence of a condition itself. Likewise, conventional predictive models may not reveal the opportunity for modifiable utilization or costs, nor the specific actions that may produce lower health care costs.

As such, there remains a need in the art of for a tool to predict future healthcare costs that provides more accurate results, as well as projections of modifiable costs and actions needed to reach the lower potential cost, than those provided by conventional approaches.

### SUMMARY

One embodiment provides a computer system, comprising: a rules engine module executed by a processor configured to electronically query a set of clinical rules representing evidence-based medical standards of care stored on a non-transitory computer readable medium; at least one network service module for receiving medical care information relating to patients, the at least one network service having real-time access to at least one source of data, including claims data containing clinical information relating to the patient; and a cost prediction module executed by processor configured to: identify a presence of one or more monitored events representing modifiable health risks based on the medical care information, wherein a monitored event is identified when the claims data indicates that the patient is receiving medical care that is not in compliance with the evidence-based medical standards of care, map each of the one or more monitored events to one or more of a utilization prediction value and a cost prediction value based on a status of each of the one or more monitored events, assign each of the one or more monitored events a probability score for utilization of medical services based on the status of each of the one or more monitored events, and calculate a prediction of future healthcare utilization and/or costs of the patient based on the probability score and the status for each of the one or more monitored events. The prediction of future healthcare utilization and/or costs can also be for a patient population.

### BRIEF DESCRIPTION OF THE DRAWINGS

FIG. 1 is a schematic illustrating an overview of a system for predicting future health care costs based on, among other things, a comparison of the best evidence-based medical standards of care to a patient's actual medical care, in accordance with one embodiment of the disclosure.

FIG. 2 is a flow chart illustrating a method for predicting future health care utilization and/or costs, in accordance with one embodiment of the disclosure.

FIG. 3 is a block diagram of basic functional components for a server or cluster of servers configured to implement embodiments of the disclosure.

FIG. 4 is a conceptual diagram showing development, validation, and evaluation samples, in one embodiment of the disclosure.

### DETAILED DESCRIPTION

Embodiments of the disclosure relate to systems and methods for predicting and estimating modifiable health care costs. According to certain embodiments, clinical features are extracted from large clinical databases (e.g., health insurance data). The clinical features are used as predictor variables in a mathematical algorithm, implemented as a statistical model, able to run against arbitrary sets of patient/health plan member data. One embodiment takes into account an evidence-based clinical decision support system (i.e., “gaps in care”) to refine the prediction of modifiable and non-modifiable utilization (i.e., cost) in the future. Each variable that corresponds to the presence or absence of a monitored event (e.g., presence or absence of a gap in care) has a coefficient, or weighting, and a status—e.g., open or closed status depending on whether a risk factor was modified. The prediction of future utilization, including not just cost but unit volume use of healthcare resources by specific categories, is therefore not only based on the presence or absence of a clinical feature or risk factor, but also the status of the modifiable clinical feature.

A health care system includes a variety of participants, including doctors, hospitals, insurance carriers, and patients. These participants frequently rely on each other for the information necessary to perform their respective roles because individual care is delivered and paid for in numerous locations by individuals and organizations that are typically unrelated. As a result, a plethora of health care information storage and retrieval systems are required to support the heavy flow of information between these participants related to patient care. Critical patient data is stored across many different locations using legacy mainframe and client-server systems that may be incompatible and/or may store information in non-standardized formats. To ensure proper patient diagnosis and treatment, health care providers must often request patient information by phone or fax from hospitals, laboratories or other providers. Therefore, disparate systems and information delivery procedures maintained by a number of independent health care system constituents lead to gaps in timely delivery of critical information and compromise the overall quality of clinical care.

Since a typical health care practice is concentrated within a given specialty, an average patient may be using services of a number of different specialists, each potentially having only a partial view of the patient's medical status. Potential gaps in complete medical records reduce the value of medical advice given to the patient by each health care provider. To obtain an overview or establish a trend of his or her medical data, a patient (and each of the patient's physicians) is forced to request the medical records separately from each individual health care provider and attempt to reconcile the piecemeal data. The complexity of medical record data also requires a significant time investment by the physician in order to read and comprehend the medical record, whether paper-based or electronic, and to ensure consistent quality of care. Additionally, while new medical research data continuously affects medical standards of care, there exists evidence of time delay and comprehension degradation in the dissemination of new medical knowledge. Existing solutions, of which there are few, have generally focused on centralized storage of health care information, but have failed to incorporate real-time analysis of a patient's health care information in order to expeditiously identify potential medical issues that may require attention and/or predict future costs. Thus, a need still exists for a computer-based solution that is capable of clinically analyzing, in real-time, the accumulated health care information in light of appropriate medical standards and directly notifying the patient and the health care provider to ensure a prompt follow up on the results of the analysis. A further need exists for a computer-based solution that is capable of clinically analyzing, in real-time, the accumulated health care information and generate a care plan for a patient. A still further need exists computer-based solution that is capable of clinically analyzing, in real-time, the accumulated health care information and generate an assessment of the deviation from the ideal evidence-based care plan, as well as a prediction of future utilization (e.g., health care costs) for a patient both as-given with current risk factors, as well as with the opportunity for decreased future health costs and utilization if the modifiable risk factors are mitigated e.g. gaps-in-care are resolved.

Some embodiments of the disclosure are used to provide an automated system for presenting a patient with an interactive personal health record powered by clinical decision support technology capable of delivering individualized alerts based on comparisons of expected medical standards of care to information related to the patient's actual medical care. Such embodiments are advantageous over previous, static health record systems that merely store and present health related information. A health care organization collects and processes a wide spectrum of medical care information in order to establish and update the relevant medical standards of care, identify the actual medical care received by the patient, and generate and deliver customized alerts, including clinical alerts and personalized wellness alerts, directly to the patient via an online interactive personal health record (PHR). The medical care information collected by the health care organization comprises patient-specific clinical data (e.g., based on claims, health care provider, and patient-entered input), as well as health reference information, including evidence-based literature relating to a plurality of medical conditions. In addition to aggregating patient-specific medical record and clinical alert information, the PHR may also solicit the patient's input for tracking of alert follow-up actions. Additionally, the PHR may accept patient-generated data including family health history, patient's allergies, current over-the-counter medications and herbal supplements, unreported and untreated conditions, as well as input for monitoring items such as blood pressure, cholesterol, and additional pertinent biometric device data and medical information that is likely to be within the realm of patient's knowledge or personal use.

A medical insurance carrier collects clinical information originating from medical services claims, performed procedures, pharmacy data, lab results, as well as structured electronic clinical data, e.g. CCD (continuity of care document) in standardized format, and provides it to the health care organization for storage in a medical database. The medical database comprises one or more medical data files located on a computer readable medium, such as a hard disk drive, solid-state storage, a CD-ROM, a tape drive, or the like.

A team of medical professionals designated by the health care organization consults various sources of health reference information, including evidence-based literature, to create and continuously revise a set of clinical rules that reflect the best evidence based medical standards of care for a plurality of conditions. The clinical rules are stored in the medical database.

The PHR facilitates the patient's task of creating a complete health record by automatically populating the data fields corresponding to the information derived from the claim, pharmacy and/or lab result-based clinical data. Preferably, the PHR gathers at least some of the patient-generated data via a health risk assessment tool (HRA) that allows direct user entry of family history, known chronic conditions and other medical data, and provides an overall patient health assessment. In some embodiments, the HRA tool presents a patient with questions that are relevant to his or her medical history and currently presented conditions. The risk assessment logic branches dynamically to relevant and/or critical questions, thereby saving the patient time and providing targeted results. The data entered by the patient into the HRA also populates the corresponding data fields within other areas of PHR and generates additional clinical alerts to assist the patient in maintaining optimum health.

The health care organization aggregates the medical care information, including the patient or nurse-entered data as well as claims data, into the medical database for subsequent processing via an analytical system. In one example, the analytical system may comprise the CareEngine® System operated by Active Health Management, Inc. of New York, N.Y.

The CareEngine® System is a multi-dimensional analytical application including a rules engine module comprising computer-readable instructions that apply a set of clinical rules reflecting the best evidence-based medical standards of care for a plurality of conditions to the patient's claims and self-entered clinical data that reflects the actual care that is being delivered to the patient. The rules engine module identifies one or more instances where the patient's actual care, as evidenced by claims data (including medical procedures, tests, pharmacy data and lab results) and patient-entered clinical data, is inconsistent with the best evidence-based medical standards of care and issues patient-specific clinical alerts directly to the patient via a set of Web pages comprising the PHR tool. Additionally, the rules engine module applies specific rules to determine when the patient should be notified, via the PHR, of newly available health information relating to their clinical profile. In one embodiment, the physician gains access to the Web pages with the consent of the patient. Still further, the rules engine module may work together with a cost predictor module to predict future health care utilization, and thus cost, based on the patient's actual care as compared to the best evidence-based medical standards of care.

In an embodiment, when the rules engine module identifies an instance of actual care inconsistent with the established, best evidence-based medical standards of care, the patient is presented with a clinical alert via the PHR. In embodiments, the clinical alerts include notifications to contact the health care provider in order to start or stop a specific medication and/or to undergo a specific examination or test procedure associated with one or more conditions and co-morbidities specific to the patient. To ensure prompt patient response, the health care organization sends concurrent email notifications to the patient regarding availability of individualized alerts at the PHR. The clinical alerts notify the patient regarding known drug interactions and suggested medical therapy based on the best evidence-based medical standards of care. In addition to condition specific alerts, the rules engine module notifies the patient regarding relevant preventive health information by issuing personalized wellness alerts, via the PHR. In one embodiment, the patient is able to use the PHR to search for specific health reference information regarding a specified condition, test or medical procedure by querying the medical database via a user interface. Preferably, the PHR allows the patient to create printable reports containing the patient's health information, including health summary and health risk assessment reports, for sharing with a health care provider.

Additionally, by functioning as a central repository of a patient's medical information, the PHR empowers patients to more easily manage their own health care decisions, which is advantageous as patients increasingly move toward consumer-directed health plans.

Further embodiments include implementing a plurality of modules for providing real-time processing and delivery of clinical alerts and personalized wellness alerts to the patient via the PHR and to a health care provider via one or more health care provider applications. Specifically, the system includes a real-time application messaging module for sending and receiving real-time information via a network between the health care organization and external systems and applications. Preferably, the real-time application messaging module employs a service-oriented architecture (SOA) by defining and implementing one or more application platform-independent software services to carry real-time data between various systems and applications.

In one embodiment, the real-time application messaging module comprises web services that interface with external applications for transporting the real-time data via a Simple Object Access Protocol (SOAP) over HTTP. The message ingest web service, for example, receives real-time clinical data that is subsequently processed in real-time by the rules engine module against the best evidence-based medical standards of care. Incoming real-time data is optionally stored in the medical database.

Incoming real-time data associated with a given patient, in conjunction with previously stored data and applicable clinical rules, defines a rules engine run to be processed by the rules engine module. Hence, the real-time application messaging module collects incoming real-time clinical data from multiple sources and defines a plurality of rules engine runs associated with multiple patients for simultaneous real-time processing.

The real-time application messaging module forwards the rules engine runs to the rules engine module to instantiate a plurality of real-time rule processing sessions. The rules engine module load-balances the rule processing sessions across multiple servers to facilitate real-time matching of the clinical rules (best evidence-based medical standards of care) against multiple, simultaneous requests of incoming clinical data and patient-entered data. When the actual mode of care for a given patient deviates from the expected mode of care, the rules engine module generates one or more clinical alerts. Similarly, the rules engine module generates real-time personalized wellness alerts based on the best evidence-based medical standards of preventive health care.

During processing, the rules engine module records alert justification information in the medical database. In one embodiment, the alert justification information specifies which clinical rules have been triggered/processed by the incoming data (e.g., by rule number), which alerts have been generated (e.g., by alert number), a time/date stamp for each alert, the specific exclusionary and inclusionary information for a given patient that caused the rule to trigger (e.g., known drug allergies are used to exclude alerts recommending a drug regimen that may cause an allergic reaction), as well as patient-entered and claim information associated with the incoming real-time data that triggered a given rule.

In yet another embodiment, the rules engine module analyzes patient specific clinical data to generate a real-time risk score for various medical conditions. The risk score quantifies the severity of existing medical conditions and assesses the risk for future conditions in light of evaluating multiple risk factors in accordance with the clinical rules. For example, the risk score may identify high risk diabetics or patients subject to a risk of future stroke. The system presents the risk score to the patient, as well as to the health care provider.

Therefore, each rule processing session produces a plurality of clinical alerts, personalized wellness alerts, and/or calculates a risk score based on a set of real-time data for a given patient. The message transmit web service, in turn, delivers the generated alerts to the PHR and/or health care provider applications. Alternatively, the application messaging module comprises a single web service for both sending and receiving real-time data. To facilitate the real-time delivery of alerts, the alert payload filtering module reduces the real-time alert payload by filtering the alert input to the real-time application messaging module by a plurality of conditions and categories. In addition to improving the speed of real-time delivery of alerts, alert filtering eliminates redundant alerts and helps to focus the recipient's attention on the important alerts.

In another embodiment, patient care management functionality is implemented. The disclosure includes querying a set of clinical rules and obtaining claims data containing clinical information relating to a plurality of patients. The system can identify patients that would benefit from care management and create a listing of markers, or conditions, associated with each identified patient based on the claims data containing clinical information relating to the patient. The system generates an individual care plan for a patient base on the identified markers, goals, problems, vision goals and the claims data containing clinical information relating to the patient.

In yet another embodiment, the systems and methods disclosed herein are used to predict future health care costs. In conventional approaches, predictive models have been used to predict healthcare costs based on static factors—e.g., age, gender, diagnoses, and medications of individuals in the populations used to develop the models. Such models have achieved modest accuracy, but do not take into account modifiable influences on health. Embodiments of the disclosure take into account how well an individual complies with evidence-based healthcare guidelines. These embodiments provide a better predictor of patients' future health care utilization and cost, compared to just knowing their demographics and clinical conditions.

As described, the CareEngine® System uses claims and lab test results to algorithmically identify diagnoses, procedures, other health-related markers and state of compliance with clinical practice guidelines on each member of a health plan or Accountable Care Organization (ACO) population. The state of these variables is updated with each run of the CareEngine® based on newly-received data, and, for each member, are output as two major categories of clinical variables called Monitored Events (ME) and Markers (MK), as will be described in detail. Although embodiments disclosed herein include data output from the CareEngine® System in one example, any system that compares health-related markers and state of compliance with clinical practice guidelines to identify the presence or absence of gaps in care may be used.

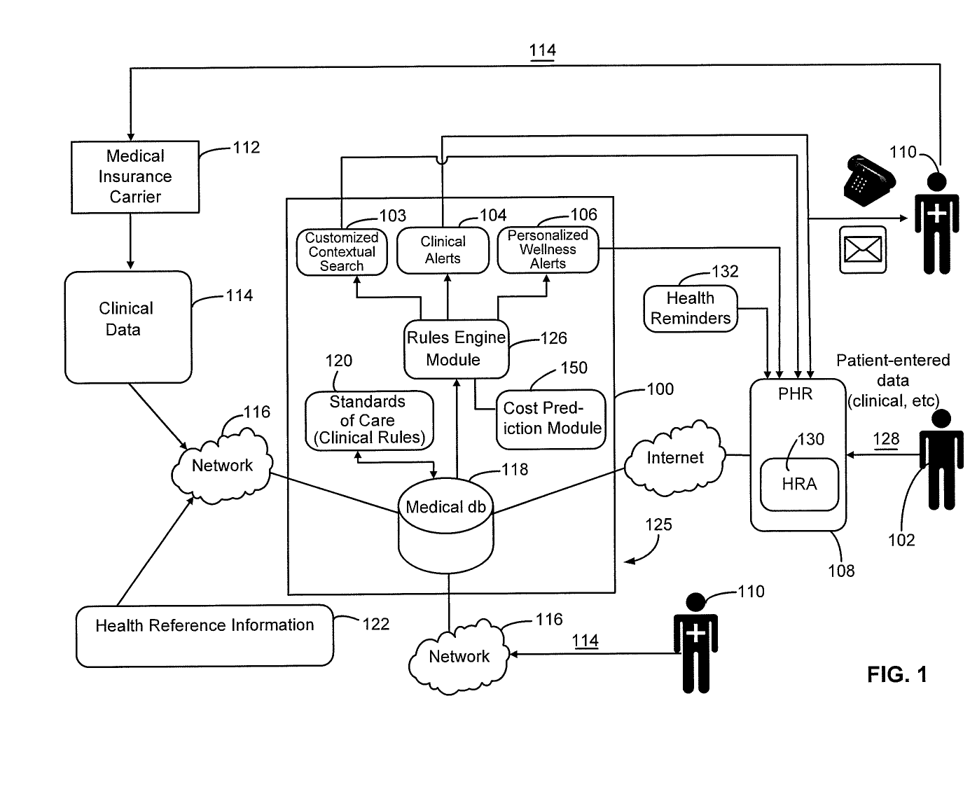

Turning to FIG. 1, an implementation of a system contemplated by embodiments of the disclosure is shown with reference to an automated system capable of predicting future health care costs based on comparison of the best evidence-based medical standards of care to a patient's actual medical care. Some embodiments also provide for presenting a patient with an interactive personal health record powered by clinical decision support technology and sending alerts to the patient.

A health care organization 100 collects and processes a wide spectrum of medical care information relating to a patient 102 in order to predict future health care costs and/or generate and deliver customized alerts, including clinical alerts 104 and personalized wellness alerts 106, directly to the patient 102 via an online interactive personal health record (PHR) 108. In addition to aggregating patient-specific medical records and alert information, as well as other functionality to be discussed herein, the PHR 108 also solicits the patient's input for entering additional pertinent medical information, tracking of alert follow-up actions and allows the health care organization 100 to track alert outcomes.

When the patient 102 utilizes the services of one or more health care providers 110, a medical insurance carrier 112 collects the associated clinical data 114 in order to administer the health insurance coverage for the patient 102. Additionally, a health care provider 110, such as a physician or nurse, enters clinical data 114 into one or more health care provider applications pursuant to a patient-health care provider interaction during an office visit or a disease management interaction. Clinical data 114 originates from medical services claims, pharmacy data, as well as from lab results, and includes information associated with the patient-health care provider interaction, including information related to the patient's diagnosis and treatment, medical procedures, drug prescription information, in-patient information and health care provider notes. The medical insurance carrier 112 and the health care provider 110, in turn, provide the clinical data 114 to the health care organization 100, via one or more networks 116, for storage in a medical database 118. The medical database 118 is administered by one or more server-based computers associated with the health care provider 100 and comprises one or more medical data files located on a computer-readable medium, such as a hard disk drive, a CD-ROM, a tape drive, or the like. The medical database 118 includes a commercially available database software application capable of interfacing with other applications, running on the same or different server-based computer via a structured query language (SQL), for example. In an embodiment, the network 116 is a dedicated medical records network. Alternatively or in addition, the network 116 includes an Internet connection that comprises all or part of the network.

An on-staff team of medical professionals within the health care organization 100 consults various sources of health reference information 122, including evidence-based preventive health data, to establish and continuously or periodically revise a set of clinical rules 120 that reflect best evidence-based medical standards of care for a plurality of conditions. The clinical rules 120 are stored in the medical database 118 or another database.

To supplement the clinical data 114 received from the insurance carrier 112, the PHR 108 allows patient entry of additional pertinent medical information that is likely to be within the realm of patient's knowledge. Exemplary patient-entered data 128 includes additional clinical data, such as patient's family history, use of non-prescription drugs, known allergies, unreported and/or untreated conditions (e.g., chronic low back pain, migraines, etc.), as well as results of self-administered medical tests (e.g., periodic blood pressure and/or blood sugar readings). Preferably, the PHR 108 facilitates the patient's task of creating a complete health record by automatically populating the data fields corresponding to the information derived from the medical claims, pharmacy data and lab result-based clinical data 114. In one embodiment, patient-entered data 128 also includes non-clinical data, such as upcoming doctor's appointments. Preferably, the PHR 108 gathers at least some of the patient-entered data 128 via a health risk assessment tool (HRA) 130 that requests information regarding lifestyle behaviors, family history, known chronic conditions (e.g., chronic back pain, migraines) and other medical data, to flag individuals at risk for one or more predetermined medical conditions (e.g., cancer, heart disease, diabetes, risk of stroke) pursuant to the processing by a rules engine module 126. Preferably, the HRA 130 presents the patient 102 with questions that are relevant to his or her medical history and currently presented conditions. The risk assessment logic branches dynamically to relevant and/or critical questions, thereby saving the patient time and providing targeted results. The data entered by the patient 102 into the HRA 130 also populates the corresponding data fields within other areas of PHR 108. The health care organization 100 aggregates the clinical data 114, patient-entered data 128, as well as the health reference information 122 including medical news, into the medical database 118 for subsequent processing via the rules engine module 126.

The CareEngine® System 125 is a multi-dimensional analytical software application including a rules engine module 126 comprising computer-readable instructions for applying a set of clinical rules 120 to the contents of the medical database 118 in order to identify an instance where the patient's 102 actual care, as evidenced by the clinical data 114 and the patient-entered data 128, is inconsistent with the best evidence-based medical standards of care. After collecting the relevant data 114 and 128 associated with the patient 102, the rules engine module 126 applies the clinical rules 120 specific to the patient's medical data file, including checking for known drug interactions, to compare the patient's actual care with the best evidence-based medical standard of care. In addition to analyzing the claims and lab result-derived clinical data 114, the analysis includes taking into account known allergies, chronic conditions, untreated conditions and other patient-reported clinical data to process and issue condition-specific clinical alerts 104 and personalized wellness alerts 106 directly to the patient 102 via a set of Web pages comprising the PHR 108. The rules engine module 126 is executed by a computer in communication with the medical database 118. In one embodiment, the computer-readable instructions comprising the rules engine module 126 and the medical database 118 reside on a computer-readable medium of a single computer controlled by the health care organization 100. Alternatively, the rules engine module 126 and the medical database 118 are interfacing via separate computers controlled by the health care organization 100, either directly or through a network.

In some embodiments, to ensure prompt patient response, the health care organization 100 may send concurrent email notifications to the patient 102 regarding availability of customized alerts 104 and 106 at the PHR 108. As described herein, the terms “alerts” and “customized alerts” refer to patient-specific health related notifications, such as clinical alerts 104 and personalized wellness alerts 106, which have been delivered directly to the patient 102 via the PHR 108 after being generated by the rules engine module 126 pursuant to the processing of one or more of the clinical data 114 and patient-entered data 128, and matched with the best evidence-based medical standards of care reflected in the clinical rules 120. In an embodiment, the alerts 104, 106 are also delivered to the health care provider 110. When the rules engine module 126 identifies an instance of actual care which is inconsistent with the best evidence-based medical standards of care, the patient 102 is presented with a clinical alert 104 via the PHR 108. Preferably, the clinical alerts 104 are prominently displayed within a user interface of the PHR 108. In embodiments, the clinical alerts 104 include notifications to contact the health care provider 110 in order to start or stop a specific medication and/or to undergo a specific test procedure associated with one or more conditions and co-morbidities specific to the patient 102. The clinical alerts 104 include notifying the patient regarding known drug interactions and suggested medical therapy derived from the current best evidence-based medical standard of care information 120. The clinical alerts 104 are also prompted by analysis of patient's medication regimen in light of new conditions and lab results. Similarly, the rules engine module 126 notifies the patient 102 regarding the clinically relevant preventive health information 122 by issuing personalized wellness alerts 106, via the PHR 108 to ensure overall consistency of care.

The rules engine module 126 also identifies the members who have at risk lifestyle behaviors (e.g., smoking, high stress, poor diet/exercise) and seeks consent from the high risk members to enroll them in a lifestyle coaching program. In one embodiment, the patient 102 is able to use the PHR 108 to search for specific health reference information regarding a specified condition, test or medical procedure by querying the medical database 118 via a user interface. In another embodiment, the patient 102 subscribes to health reference information 124 for delivery via the PHR 108 and/or personal email. In yet another embodiment, the rules engine module 126 automatically generates a customized contextual search 103 of the health reference information 122 and/or an external source of medical information, based on the patient's clinical data 114 and patient-entered data 128 for delivery of the search results via the PHR 108. In yet another embodiment, the patient 102 receives general health reminders 132 based on non-clinical components of the patient-entered data 128 that are not processed by the rules engine module 126, such as notifications regarding upcoming doctor appointments. In embodiments, general health reminders 132 include prompting the patient 102 to update the HRA 130, watch a video tour of the PHR website, or update the health tracking information (discussed below in connection with FIG. 16). Preferably, the PHR 108 allows the patient 102 to create printable reports containing the patient's health information, including health summaries and health risk assessment reports, for sharing with the health care provider 110.

To ensure further follow-up, the health care organization 100 optionally notifies the health care provider 110 regarding the outstanding clinical alerts 104. For example, if a clinical alert 104 includes a severe drug interaction, the health care organization 100 prompts the health care provider 110, via phone, mail, email or other communications, to initiate immediate follow-up.

In one embodiment, the rules engine module 126 interacts with a utilization and/or cost prediction module 150 that can predict future health care utilization and/or costs based on, among other things, a comparison of the best evidence-based medical standards of care to a patient's actual medical care. According to certain embodiments, clinical features called “markers” and “monitored events” are extracted from large clinical databases (e.g., health insurance data). “Monitored events” are a set of clinical sub-features that represent a modifiable health risk or clinical scenario likely to predispose to clinical risk and resultant utilization and cost. Monitored Events are further assigned to specific health actions that may be performed by clinicians and/or patients to mitigate a modifiable health risk. The modifable clinical features are used as predictor variables in a mathematical algorithm, implemented as a statistical model, able to run against arbitrary sets of patient/health plan member data. One embodiment takes into account an evidence-based clinical decision support system (i.e., gaps in care) to refine the prediction of utilization (e.g. hospital admissions, emergency visits, and/or associated costs) in the future. Each variable that corresponds to the presence or absence of a monitored event (i.e., gap in care) has a coefficient, or weighting, and a status—e.g., open or closed status, as described in greater detail below. A set of models based on the candidate clinical feature variables predicts a set of medical cost and utilization categories, e.g. hospital inpatient admissions, emergency department visits, outpatient primary care, outpatient specialty care, outpatient surgical procedures, pharmacy, specialty pharmacy, medical pharmacy, laboratory utilization, imaging and radiology, and mental health related costs.

In one embodiment, “markers” refer to a category of findings that refer to clinical attributes, rather than tasks/actions/events driven. Examples of markers include: presence of a given chronic disease (e.g., diabetes, asthma, heart failure), pre-disease state (e.g., pre-diabetes, pre-hypertension, metabolic syndrome), gender, age, height, weight, demographic information, etc. “Monitored events,” on the other hand, refer to clinical items associated with a modifiable health risk that may have a status, such as open or closed status. Monitored events include (a) “care considerations,” which are evidence-based medicine gaps in care that are discovered in the administrative claims, pharmacy & lab data, and (b) “health actions,” a superset of care considerations, which may include non-evidence-based events that have a status.

Examples of monitored events include: adding a drug (e.g., for a diabetic with early nephropathy who needs to be on a drug called an ACE-inhibitor to prevent future kidney failure), stopping a drug (e.g., a drug-drug interaction between a male enhancement drug and nitrates, or a drug-condition interaction between a migraine drug called triptan and coronary artery disease), adding a test (e.g., a patient taking a statin drug who needs their liver checked as a routine test), doing a work-up (e.g., for a patient with a constellation of symptoms suggesting a medical syndrome, e.g. celiac sprue, but without evidence of the diagnosis nor diagnostic testing).

Examples of health actions include some other types of medical scenarios—and are typically directed at members directly, e.g. achieve condition target, such as a specific blood pressure target.

The monitored events, which include care considerations, are composed of components called medical findings. One intent of the embodiments of the disclosure is to use these monitored events and the substituent medical findings as candidate predictor variables for future health care utilization (by unit volume) and/or costs.

While the entity relationships described above are representative, those skilled in the art will realize that alternate arrangements are possible. In one embodiment, for example, the health care organization 100 and the medical insurance carrier 112 is the same entity. Alternatively, the health care organization 100 is an independent service provider engaged in collecting, aggregating and processing medical care data from a plurality of sources to provide a personal health record (PHR) service for one or more medical insurance carriers 112. In yet another embodiment, the health care organization 100 provides PHR services to one or more employers by collecting data from one or more medical insurance carriers 112.

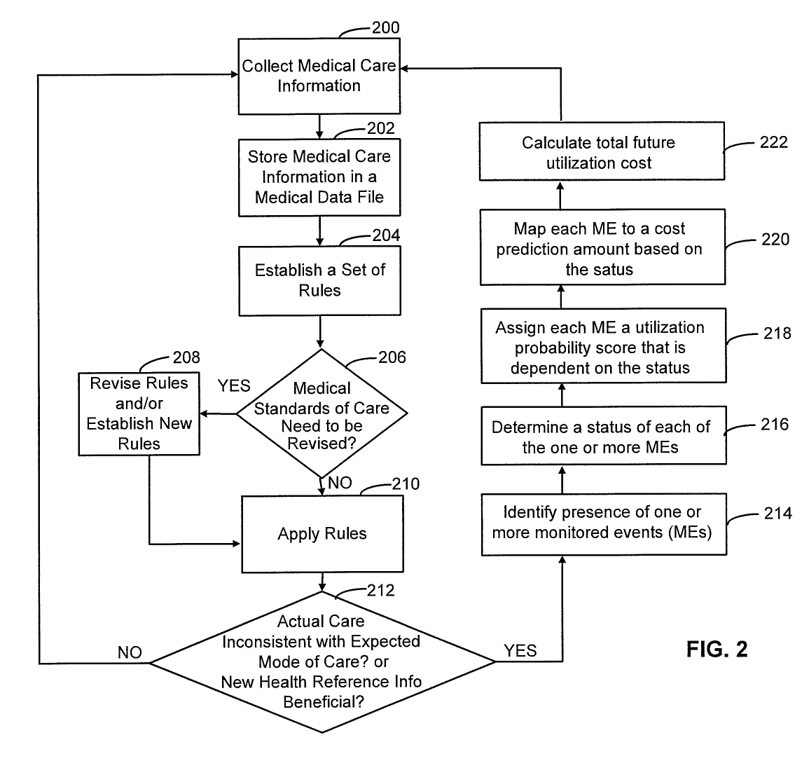

Turning to FIG. 2, a method for predicting future health care utilization and/or costs, in accordance with one embodiment of the disclosure, is described. In step 200, a health care organization 100 collects a wide spectrum of medical care information 114, 122, 124, 128, and, at step 202, the health care organization 100 stores the medical care information in a medical database 118 for subsequent analysis. In step 204, the health care organization 100 establishes a set of clinical rules 120 for a plurality of conditions, such as by having a medical professional team continuously review collected health reference information 122, including evidence-based medical literature. In steps 206-208, when updates to the medical standards of care become available (e.g., upon collecting additional or updated evidence-based literature), the health care organization 100 revises the clinical rules 120 and builds new rules associated with the best evidence-based medical standards of care.

In steps 210 and 212, a rules engine module 126 applies the latest evidence-based medical standards of care included within the clinical rules 120 to the patient's actual care, as evidenced from the clinical data, claims, pharmacy, lab and patient-entered clinical data, to identify at least one instance where the patient's actual care is inconsistent with the expected care embodied by the clinical rules 120. Step 212 also optionally includes identifying whether the patient 102 should be notified about newly available evidence-based standards of preventive health care 122 via a personalized wellness alert, such as when the preventive health care information is beneficial to the patient's actual care (e.g., notifications regarding the benefits of breast cancer screening).

If the rules engine module 126 does not detect a discrepancy between the actual care given by the caregiver and the best evidence-based medical standards of care, or when the newly received health reference is not beneficial (e.g., cumulative in light of existing information), the method returns to step 200.

If, at step 212, the rules engine module 126 does detect a discrepancy between the actual care given by the caregiver and the best evidence-based medical standards of care, the method proceeds to step 214. At step 214, the utilization and/or cost prediction module 150 identifies the presence of one or more monitored events. As described, monitored events refer to modifiable clinical items that may have a status associated with the modifiable clinical item. As an example, if a patient is diabetic, the evidence-based standard of care may recommend for the patient to get an eye exam. If the patient does not get an eye exam, then the patient is not in compliance with the evidence-based standard of care. In such an example, the status of the monitored event remains open. There can be many different status identifiers for whether the patient has had the recommended eye exam, not merely open or closed, as is outlined below.

At step 216, the utilization and/or cost prediction module 150 determines a status of each of the one or more monitored events. Continuing with the example of a diabetic patient who is recommended to get an eye exam, some example statuses are provided below. A “completion” status may identify that the monitored event been successfully completed. For example, the status may be “open” if the patient has not taken an eye exam, and may be “closed” if the patient did get an eye exam. A “denominator” status identifies whether the monitored event should even apply to this patient at all (e.g., is the patient a non-blind diabetic). A “retraction” status identifies that the data suggests that the event is invalid or has never applied to this patient (e.g., we discover the patient was born without eyes, so getting an eye exam is not applicable). A “historic” status identifies that the monitored event is now obsolete or no longer applies to the patient, but may have applied in the past (e.g. we discover the patient recently lost their sight or eyes). For some monitored events, the concept of “denominator” does not apply. For example, drug-drug interactions do not have a denominator per se—for example, some embodiments do not measure everyone taking the drug, and then look at who is not taking an interacting drug.

In some embodiments, medical findings are tagged to the individual members by applying a clinical rule. The rule is a boolean-style expression that refers to a proprietary dictionary of code groupings (SNOMED, ICD-9, CPT, NDC, LOINC, etc.). The rule may also incorporate sub-calculations (e.g., kidney function as estimated glomerular filtration rate; staging of disease). The code groupings are called ELEMENTS. Individual codes within the ELEMENTS are called ATOMS. Therefore, as the CareEngine® runs on the data for a given population, it tags members with medical findings, which are then rolled-up and collapsed into MEDICAL FINDINGS with specific statuses.

Additionally, in some embodiments, there is another type of medical finding called a “safety alert” that has less to do with a clinical scenario where the status may change, and more to do with flagging patients/members for a specific notification. This is typically related to newly-announced black box warnings from the FDA. Thus, there is not necessarily a specific immediate action, per se, for the provider or patient other than to be aware of new side-effects or adverse events. Such safety alert medical findings contribute to a monitored event type called “patient safety alert,” and may also be accompanied by patient-generated data opportunity to report symptoms or signs of an adverse event or side effect through a patient portal or personal health record interface, to be added to that patient's medical data in the database.

At step 218, the utilization and/or cost prediction module 150 assigns, to each of the one or more monitored events that is present, a probability score corresponding to a probability of utilization based, where the probability is based on the status of the monitored event. In some embodiments, based on the defined schema, each of the monitored events (MEs) falls under a care consideration (CC). Some CCs have a single ME. In some embodiments, a joint probability score is computed for each CC, based on the set of MEs constituting such care consideration group. Below is a description of the methodology.

First, embodiments of the disclosure assign each ME a probability score, which is the probability of a utilization event in presence of that ME. This is a conditional probability given by the equation:

$${PME}_{ij} = P ( X = 1 \vert {ME}_{ij} = 1 ) = \frac{Members with X = 1 and {ME}_{ij} = 1}{Members with {ME}_{ij} = 1} ,$$

where X and MEᵢⱼ are flags on specific-utilization and monitored event, respectively. The index i is for ME, and j for its status (e.g., open/closed/etc.). Thus, PMEᵢⱼ is the probability score for the i^{th} ME with j^{th} status. The above formula gives the fraction of members with a given monitored event and an event status who had a claim for utilization event.

In some embodiments, the above probability scores are computed for each ME status separately, e.g., computed for both open and closed ME status. Hence, for any ME, there will be two or more probability scores. For example, one score may be based on members with an open status of the ME and another score other with the closed status.

In some embodiments, the probability score computation is only performed to those MEs that have a minimum number of members having that ME, e.g., more than 100 members. This cutoff is applicable on open and closed status members separately. Hence, some MEs might have only one probability score for either open or closed status, as the frequency of the other status members was below the threshold. The selection of the at least 100 members is merely an example, but experiments show that this number may help to avoid cases that had every few observations, and thus not enough statistical significance.

Once each ME is assigned a probability scores for each status of the ME, a joint score can be computed using the probability scores of the constituent MEs in a care consideration (CC). As described, in some embodiments each CC maps to certain MEs (e.g., several MEs may map to one CC). The joint probability score for a CC (having several monitor events such as ME₁, ME₂, . . . , MEₙ) is given by the equation:

PCCₖⱼ=1−(1−PME₁ᵢ)(1−PME₂ⱼ) . . . (1−PMEₙⱼ),

where PMEᵢⱼ is the probability score for MEᵢ with event status j. The index i is for CC and j for the status of MEs. The joint probability score quantifies the probability that at least one of the MEs will contribute to the occurrence of utilization. In some embodiments, a joint probability score is computed for a CC only if it has more than one MEs with a probability score, i.e., at least two MEs with frequencies over 100. Each CC has two joint probability scores, one for MEs with open status and the other for closed.

At step 220, the utilization and/or cost prediction module 150 maps each monitored event to a utilization and/or cost prediction based on the status of the monitored event. For example, for a given ME, a cost of $100 may be assigned to the ME if the status of the ME is open, whereas a cost of $50 may be assigned to the ME if the status of the ME is closed. For each ME and status combination, a different utilization and/or cost prediction may be utilized. In some embodiments, the cost amounts may be dependent on contracted discounts.

At step 222, the utilization and/or cost prediction module 150 predicts the total future utilization of medical services. In some embodiments, the total predicted value is computed by calculating a sum of the individual ME-status costs identified at step 220. The method then returns to step 200.

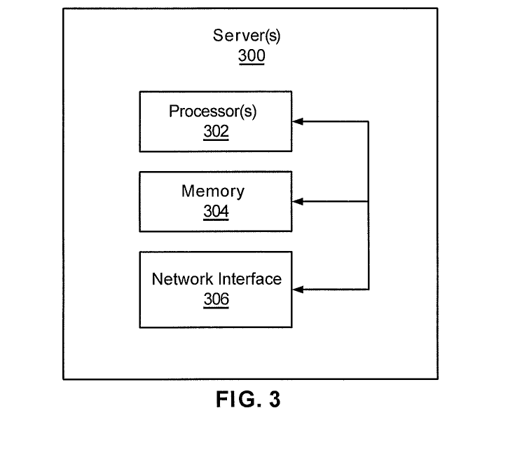

FIG. 3 is a block diagram of basic functional components for a server or cluster of servers configured to implement embodiments of the disclosure. For example, server 300 may represent the computer system that executes the rules engine module 126 and the utilization and/or cost prediction module 150 shown in FIG. 1. In some embodiments, server 300 is configured to be a computer or computers operated by an insurance carrier.

The server 300 includes one or more processors 302, memory 304, and network interface 306. In some embodiments, each of the components including the processor(s) 302, memory 304, and network interface 306 is interconnected physically, communicatively, and/or operatively for inter-component communications.

As illustrated, processors 302 are configured to implement functionality and/or process instructions for execution within server 300. For example, processors 302 execute instructions stored in memory 304. Memory 304, which may be a non-transient, computer-readable storage medium, is configured to store information within server 300 during operation. In some embodiments, memory 304 includes a temporary memory, i.e., an area for information not to be maintained when the server 300 is turned off. Examples of such temporary memory include volatile memories such as random access memories (RAM), dynamic random access memories (DRAM), and static random access memories (SRAM). Memory 304 also maintains program instructions for execution by the processors 302.

The server 300 uses network interface 306 to communicate with external devices via one or more networks, such as the network 116 in FIG. 1. Such networks may include one or more cellular networks, wireless networks, wired networks, fiber optics networks, and other types of networks through which communication between the server 300 and an external device may be established. Network interface 306 may be a network interface card, such as an Ethernet card, an optical transceiver, a radio frequency transceiver, or any other type of device that can send and receive information.

As mentioned above, the server 300 is configured to predict future health care costs based on, among other things, a comparison of the best evidence-based medical standards of care to a patient's actual medical care, as disclosed herein. According to certain embodiments, the server 300 is configured to receive medical information from medical database 118 and execute the rules engine module 126 and the utilization and/or cost prediction module 150 shown in FIG. 1.

In some embodiments, the utilization and/or cost prediction module 150 retrieves data from multiple data sources in real-time (i.e., simultaneously or nearly simultaneously) to perform its calculations. For example, evidence-based medial rules may be retrieved from a first database, and medical claims data may be retrieved from a second database. The data from the disparate sources is then analyzed together in real-time. Also, in some embodiments, the medical claims data in the second database may be continuously updated with new medical claims data from each of a large number of individuals that is present in the dataset. As such, the analysis performed by the utilization and/or cost prediction module 150 of comparing the evidence-based medial rules to the medical claims data may be ongoing and continuous, such that new analysis is performed by the utilization and/or cost prediction module 150 in real-time as new claims data is generated or at some predefined interval. In some embodiments, because of the vast amount of information being analyzed, and because the data is derived from multiple and varied data sources, the disclosed embodiments could not be performed outside of a computerized environment.

Various techniques may be implemented to generate the model used to predict future costs in accordance with embodiments of the disclosure. In one implementation, separate predictive models may be created for each major claims utilization category. For example, one model will predict the probability of having at least one in-patient hospitalization for each member. Another model may be developed for primary care physician (PCP) office visits to predict the frequency of such visits for each member.

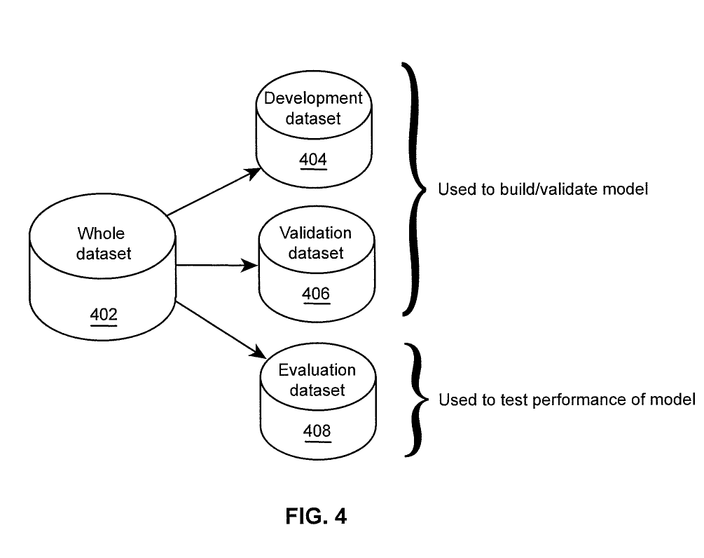

In one implementation, for each model, the member level dataset can be split into development, validation, and evaluation samples, as shown in FIG. 4. From a dataset 402 (e.g., which may comprise a historical dataset of claims data for a given year), a development dataset 404 is used to build the model and a validation dataset 406 is used to fine tune the model. When fine-tuning the model, some embodiments investigate the robustness of the parameter estimates and their changes from one sample to another. When this step is finalized, both samples are combined back and the model is re-executed to refresh the parameters estimate. Both the development dataset 404 and validation dataset 406 are used to build the model; therefore, they cannot be used as evaluation set. However, the third data split, i.e., an evaluation dataset 408, is not used during the model development. The evaluation dataset 408 is kept as a test dataset for evaluation of the model performance.

For the proposed models representing separate claims utilization categories, some embodiments investigated the appropriate underlying data distribution for different utilization measures before fitting any specific model. This consideration included a large class of distributions, such as normal and generalized normal distributions, including Logistic, Poisson, and Negative Binomial distributions. Some embodiments also include the number of members with zero utilization. The normal distribution can be used as a possible approximation for underlying count of certain utilization. A Logistic regression model could predict the occurrence of (at least one) utilization in a given year for a member, while the Poisson or Negative Binomial regression model predicts the count of utilization.

In some embodiments, the independent variables include both monitored events (“ME”) (i.e., clinical features) and markers (“MK”) (e.g., dememographic and non-medical type variables), as well as some variables derived from existing variables. Demographic variables have information such as age, gender, socioeconomic status (based on zip code), and region, etc. In addition to those existing variables, there are several other derived variables, such as “ME_counts”, “MK_counts”, “age-square” and “Acute_flag”.

Acute_flag refers to those MEs that are identified as having different monitoring nature compared to rest of the MEs. As a result, a flag is defined to indicate which ME falls under acute class. The word “acute” here does not necessary reflect “clinical” acuity, and may be used as a term used to classify existing and over 1,000 MEs into two separate groups. This simple classification, however, was found to be very useful to group MEs as predictors for some models.

Several interaction terms were also introduced as independent variables into the model, such as “Age*Total_acute_ME”, “Gender*Total_acute_ME”. Table 1 below shows an example list of transformations and created fields.

TABLE 1

| | SOURCE | | |
|---|---|---|---|
| TYPE | FIELD | TRANSFORMATION | GENERATED FIELDS |
| Care Engine | Monitored | Flags on each ME for both | ME_Y_xxx, ME_N_xxx, |
| | Events | open and closed statuses | ME_count |
| | | Flag on members with an | Acute_flag, |
| | | acute ME | Acute_ME_count, |
| | | | Non_acute_ME_count |
| | | CC joint probability | CC_Y_xxx, CC_N_xxx |
| | Condition | Flag on each marker | MK_xxx, MK_count |
| | Markers | | |
| Demography | Age | Square of age | Age_sq |
| | Gender | Flag on female gender | Tot_female |
| | SES | Flags on intervals | SES_{—} |
| Interaction | Monitored | Total_acute_me*tot_age | Total_acute_me*tot_age |
| | Events, Age | | |
| Interaction | Monitored | Total_acute_me*tot_female | Total_acute_me*tot_female |
| | Events, Gender | | |

For all the models developed, we considered several goodness-of-fit criteria as performance measures for the developed models. As previously indicated, the performance measures are derived using the evaluation dataset 408, i.e., the data set that was hold to test the quality of the model. The model can be fine-tuned to provide a prediction that most closely resembles actual utilization, as compared to historical data.

All references, including publications, patent applications and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method, comprising:
electronically querying, by a rules engine module executed by a computer system, a set of clinical rules representing evidence-based medical standards of care stored on a non-transitory computer readable medium;
interfacing with at least one network service for receiving medical care information relating to patients, the at least one network service having real-time access to at least one source of data, including claims data containing clinical information relating to the patient;
identifying, by a cost prediction module executed by the computer system, a presence of one or more monitored events representing modifiable health risks based on the medical care information, wherein a monitored event is identified when the claims data indicates that the patient is receiving medical care that is not in compliance with the evidence-based medical standards of care;
mapping, by the cost prediction module, each of the one or more monitored events to one or more of a utilization prediction value and a cost prediction value based on a status of each of the one or more monitored events;
assigning, by the cost prediction module, each of the one or more monitored events a probability score for utilization of medical services based on the status of each of the one or more monitored events; and
calculating a prediction of future healthcare utilization and/or cost of the patient based on the probability score and the status for each of the one or more monitored events.

2. The method of claim 1, wherein a monitored event is based on a comparison of evidence-based medical standards of care to the patient's actual medical care.

3. The method of claim 1, wherein a status of the monitored event is open or closed.

4. The method of claim 1, wherein for a first monitored event, a status of the first monitored event identifies that the first monitored event has been successfully completed.

5. The method of claim 1, wherein for a first monitored event, a status of the first monitored event identifies that the first monitored event should not apply to the patient.

6. The method of claim 1, wherein for a first monitored event, a status of the first monitored event identifies that the first monitored event is now obsolete or no longer applies to the patient.

7. The method of claim 1, further comprising grouping two or more monitored events into a care consideration, wherein a probability score for utilization of medical services based on the status of the care consideration, and calculating the prediction of future healthcare utilization cost of the patient is based on the probability score for the care consideration.

8. The method of claim 1, wherein the steps of electronically querying the set of clinical rules stored on the non-transitory computer readable medium, interfacing with at least one network service for receiving medical care information relating to the patients, and calculating the prediction of future healthcare utilization cost of the patient are performed in real-time or near real-time.

9. The method of claim 1, wherein medical care information relating to patients is received from a claims database and/or an electronic patient health record.

10. A non-transitory computer-readable storage medium storing instructions that, when executed by a computer system, cause the computer system to perform the following steps:
electronically querying, by a rules engine module executed by the computer system, a set of clinical rules representing evidence-based medical standards of care stored on a non-transitory computer readable medium;
interfacing with at least one network service for receiving medical care information relating to patients, the at least one network service having real-time access to at least one source of data, including claims data containing clinical information relating to the patient;
identifying, by a cost prediction module executed by the computer system, a presence of one or more monitored events representing modifiable health risks based on the medical care information, wherein a monitored event is identified when the claims data indicates that the patient is receiving medical care that is not in compliance with the evidence-based medical standards of care;
mapping, by the cost prediction module, each of the one or more monitored events to one or more of a utilization prediction value and a cost prediction value based on a status of each of the one or more monitored events;
assigning, by the cost prediction module, each of the one or more monitored events a probability score for utilization of medical services based on the status of each of the one or more monitored events; and
calculating a prediction of future healthcare utilization and/or cost of the patient based on the probability score and the status for each of the one or more monitored events.

11. The computer-readable storage medium of claim 10, wherein a monitored event is based on a comparison of evidence-based medical standards of care to the patient's actual medical care.

12. The computer-readable storage medium of claim 10, wherein a status of the monitored event is open or closed.

13. The computer-readable storage medium of claim 10, wherein for a first monitored event, a status of the first monitored event identifies that the first monitored event has been successfully completed.

14. The computer-readable storage medium of claim 10, wherein for a first monitored event, a status of the first monitored event identifies that the first monitored event should not apply to the patient.

15. The computer-readable storage medium of claim 10, wherein for a first monitored event, a status of the first monitored event identifies that the first monitored event is now obsolete or no longer applies to the patient.

16. The computer-readable storage medium of claim 10, further comprising grouping two or more monitored events into a care consideration, wherein a probability score for utilization of medical services based on the status of the care consideration, and calculating the prediction of future healthcare utilization cost of the patient is based on the probability score for the care consideration.

17. The computer-readable storage medium of claim 10, wherein the steps of electronically querying the set of clinical rules stored on the non-transitory computer readable medium, interfacing with at least one network service for receiving medical care information relating to the patients, and calculating the prediction of future healthcare utilization cost of the patient are performed in real-time or near real-time.

18. A computer system, comprising:
a rules engine module executed by a processor configured to electronically query a set of clinical rules representing evidence-based medical standards of care stored on a non-transitory computer readable medium;
at least one network service module for receiving medical care information relating to patients, the at least one network service having real-time access to at least one source of data, including claims data containing clinical information relating to the patient; and
a cost prediction module executed by processor configured to:
identify a presence of one or more monitored events based on the medical care information, wherein a monitored event representing modifiable health risks is identified when the claims data indicates that the patient is receiving medical care that is not in compliance with the evidence-based medical standards of care,
map each of the one or more monitored events to one or more of a utilization prediction value and a cost prediction value based on a status of each of the one or more monitored events,
assign each of the one or more monitored events a probability score for utilization of medical services based on the status of each of the one or more monitored events, and
calculate a prediction of future healthcare utilization and/or cost of the patient based on the probability score and the status for each of the one or more monitored events.

19. The computer system of claim 18, wherein a status of the monitored event is open or closed.

20. The computer system of claim 18, wherein for a first monitored event, a status of the first monitored event:
identifies that the first monitored event has been successfully completed; or
identifies that the first monitored event should not apply to the patient; or
identifies that the first monitored event is now obsolete or no longer applies to the patient.

21. The computer system of claim 18, wherein the steps of electronically querying the set of clinical rules stored on the non-transitory computer readable medium, interfacing with at least one network service for receiving medical care information relating to the patients, and calculating the prediction of future healthcare utilization cost of the patient are performed in real-time or near real-time.
